(19) United States
(12) Patent Application Publication
(10) Pub. No.: US 2019/0201692 A1
(43) Pub. Date: Jul. 4, 2019

(54) TREATMENT OF CONDITIONS ASSOCIATED WITH IMPAIRED GLUCOSE CONTROL

(71) Applicants: Galvani Bioelectronics Limited, Brentford, Middlesex (GB); The Johns Hopkins University, Baltimore, MD (US)

(72) Inventors: Arun SRIDHAR, Stevenage, Hertfordshire (GB); Liansheng LIU, Baltimore, MD (US); Pankaj Jay PASRICHA, Baltimore, MD (US)

(21) Appl. No.: 16/302,941

(22) PCT Filed: May 19, 2017
(86) PCT No.: PCT/EP2017/062193

§ 371 Date: Nov. 19, 2018

Related U.S. Application Data
(60) Provisional application No. 62/339,220, filed on May 20, 2016.

(51) Int. Cl. A61N 1/36

(52) U.S. Cl. CPC A61N 1/36034; A61N 1/36085; A61N 1/36017; A61N 1/36171

(57) ABSTRACT

A method for treating conditions associated with impaired glucose control, involving inhibition of signalling by the greater splanchnic nerve leads to a significant improvement in oral glucose tolerance.

### FIELD OF THE INVENTION

This invention relates to medical devices for the treatment of metabolic syndrome conditions, more particularly to medical devices that deliver neuromodulatory therapy for such purposes.

### BACKGROUND OF THE INVENTION

The increase in prevalence of metabolic disorders such as type 2 diabetes mellitus (T2D), obesity, and impaired glucose tolerance (where patients go on to develop T2D if left untreated) constitutes a severe unmet medical need. Moreover, they can exist in combination, and metabolic syndrome is a clustering of at least three of five medical conditions: abdominal obesity; elevated blood pressure; elevated fasting plasma glucose; high serum triglycerides; and low high-density lipoprotein (HDL) levels. Metabolic syndrome is associated with the risk of developing cardiovascular disease and diabetes.

Known treatments for these disorders are based on administration of pharmaceuticals, but these treatments are often unable to control the disease, and may produce unwanted side effects.

Gastric bypass surgery has revealed a critical role for the duodenum in glycemic/metabolic control but the exact mechanisms remain poorly understood. Previous studies have shown that duodenal spinal afferents expressing TRPV1 are important in mediating the post-prandial glycemic response, and neuromodulation has been proposed as a way of treating T2D. For instance, patent application US-2014/0187619 reports that ablation of sensory nerves in the duodenum can provide treatment for T2D, whereas US-2008/0312714 has proposed that electrical stimulation of the liver can provide a similar effect.

WO2016/072875 discloses that modulation of neural activity in the carotid sinus nerve can treat conditions associated with impaired glucose control.

There remains a need for further and improved treatments of metabolic disorders which involve impaired glucose control, such as T2D.

### SUMMARY OF THE INVENTION

The present inventors have assessed the impact of neuromodulation of glucose tolerance in animal models of diabetes and have demonstrated that inhibition of neural activity of the greater splanchnic nerve (GSN) leads to a significant improvement in oral glucose tolerance.

Thus the invention provides a method of treating a condition associated with impaired glucose control in a subject by inhibiting neural activity in the subject's GSN. A preferred way of inhibiting GSN activity uses a device which applies a signal to the GSN as described elsewhere herein.

The invention also provides a method of treating a condition associated with impaired glucose control in a subject, comprising a step of applying a signal to the subject's GSN to inhibit the neural activity of the GSN in the subject.

The invention also provides a device or system for inhibiting the neural activity of a subject's GSN, the device or system comprising (i) one or more transducers configured to apply a signal to the GSN and (ii) a controller coupled to the one or more transducers, the controller controlling the signal to be applied by the one or more transducers, such that the signal inhibits the neural activity of the GSN in order to provide an improvement in a measurable physiological parameter (particularly in a subject having a condition associated with impaired glucose control).

The invention also provides a method of treating a condition associated with impaired glucose control in a subject, comprising: implanting in the subject a device or system of the invention; positioning at least one transducer of the device/system in signalling contact with the subject's GSN; and activating the device/system.

Similarly, the invention provides a method of inhibiting neural activity in a subject's GSN, comprising: implanting in the subject a device/system of the invention; positioning at least one transducer of the device/system in signalling contact with the subject's GSN; and activating the device/system.

Further, the invention provides a method of implanting a device/system of the invention in a subject, comprising a step of: positioning at least one transducer of the device/system in signalling contact with the subject's GSN. In some embodiments the method includes a step of activating the device/system; in other methods, this activation step does not occur.

The invention also provides a device/system of the invention, wherein the device/system is attached to a GSN as described herein.

The invention further provides a neuromodulatory electrical waveform for use in treating a condition associated with impaired glucose control in a subject, wherein the waveform is a kiloHertz alternating current (AC) waveform having a frequency of 1 to 50 KHz, optionally 25-50 kHz, such that, when applied to a subject's GSN, the waveform inhibits neural activity in the GSN.

The invention also provides the use of a neuromodulation device or system for treating a condition associated with impaired glucose control in a subject, by inhibiting neural activity in the subject's GSN.

The invention also provides a GSN to which a transducer of a device/system of the invention is attached. The GSN is ideally present in situ in a subject.

The invention also provides a modified GSN to which a transducer of the system or device of the invention is attached. The transducer is in signaling contact with the nerve and so the nerve can be distinguished from the nerve in its natural state. Furthermore, the nerve is located in a subject who suffers from a condition associated with impaired glucose control.

The invention also provides a modified GSN, wherein the neural activity is reversibly inhibited by applying a signal to the GSN.

The invention also provides a modified GSN, wherein the nerve membrane is reversibly depolarised or hyperpolarised by an electric field, such that an action potential does not propagate through the modified nerve.

The invention also provides a modified GSN bounded by a nerve membrane, comprising a distribution of potassium and sodium ions movable across the nerve membrane to alter the electrical membrane potential of the nerve so as to propagate an action potential along the nerve in a normal state; wherein at least a portion of the nerve is subject to the application of a temporary external electrical field which modifies the concentration of potassium and sodium ions within the nerve, causing depolarisation or hyperpolarisation of the nerve membrane, thereby temporarily blocking the propagation of the action potential across that portion in a disrupted state, wherein the nerve returns to its normal state once the external electrical field is removed.

The invention also provides a charged particle for use in a method of treating a condition associated with impaired glucose control in a subject, wherein the charged particle causes reversible depolarisation or hyperpolarization of the nerve membrane of the GSN, such that an action potential does not propagate through the modified nerve.

The invention also provides a modified GSN obtainable by reversibly inhibiting neural activity of the GSN according to a method of the invention.

The invention also provides a method of modifying a GSN's activity, comprising a step of applying a signal to the GSN in order to inhibit the neural activity of the GSN in a subject.

The invention also provides a method of controlling a device/system of the invention which is in signalling contact with a GSN, comprising a step of sending control instructions to the device/system, in response to which the device/system applies a signal to the GSN.

The invention also provides a diabetes medicine for use in treating a subject, wherein the subject has an implanted device/system of the invention in signalling contact with their GSN.

### BRIEF DESCRIPTION OF THE DRAWINGS

FIG. 1 Example implementations of a neuromodulation device for carrying out the invention.

FIG. 2 shows OGTT scores (blood glucose tolerance, area under curve) in rats on a normal diet (ND) or a high fat diet (HFD), who had a GSN transection (-GSN) or a sham operation (-sham). The x-axis shows a pre-treatment score, then scores at 1, 4, 8, and 12 weeks after the operation. The symbol ‘*’ indicates a statistically significant difference between the -GSN and -sham animals on the same diet, whereas ‘#’ indicates a statistically significant difference between the HD and HFD animals who received the same treatment.

FIG. 3 similarly shows scores from OGTT in diabetic rats (ZDF) or a lean rats (ZDF-lean) who had a GSN transection (GSN cut) or a sham operation (Sham). The x-axis shows a pre-treatment score, then scores at 1, 4, and 8 weeks after the operation. The symbol ‘*’ indicates a statistically significant difference between the -GSN and -sham animals in the same strain, whereas ‘#’ indicates a statistically significant difference between the diabetic and lean animals who received the same treatment.

FIG. 4 shows blood glucose levels (mg/dl) in rats in an intraperitoneal glucose tolerance test in diabetic rats (ZDF) or a lean rats (ZDF-lean) who had a GSN transection (GSN cut) or a sham operation (Sham). The x-axis shows a pre-treatment score, then scores at 10, 30, 60, 90 and 120 minutes after the injection of glucose. The symbols ‘*’ and ‘#’ mean the same as FIG. 3.

FIG. 5 shows blood glucose levels (mg/dl) in rats after injection of insulin. The groups are the same as those in FIGS. 3 & 4.

FIG. 6 illustrates the positions of possible sites (1), (2), (3) and (4) in the GSN for inhibiting signalling. The abdominal artery, celiac ganglion and suprarenal ganglion are labelled, and the kidney and adrenal gland are both visible.

FIG. 7 shows blood pressure (mmHg) in ND and HFD rats (same groups and symbols as FIG. 2). FIG. 7A shows diastolic pressure, whereas 7B shows systolic pressure.

FIG. 8 shows blood pressure (mmHg) in ND and HFD rats (same groups and symbols as FIG. 3). FIG. 8A shows diastolic pressure, whereas 8B shows systolic pressure.

### DETAILED DESCRIPTION OF THE INVENTION

The inventors have shown that interruption of GSN activity in rat models of metabolic syndromes leads to significant improvements in oral glucose tolerance, as well as significantly lower insulin levels in response to glucose challenge. Thus duodenal innervation plays a role in the pathogenesis of obesity-induced type 2 diabetes, insulin resistance and hypertension, which provides a rationale for using bioelectronics (or other approaches) to inhibit GSN activity and thereby achieve therapeutic effects.

In general, a subject of interest for the invention is a human being, and in particular a human suffering from a condition associated with impaired glucose control. In pre-clinical and experimental settings, however, the invention can also extend to non-human mammals.

### The Greater Splanchnic Nerve

The splanchnic nerves carry fibers of the autonomic nervous system (visceral efferent fibers) and sensory fibers from various organs (visceral afferent fibers). All splanchnic nerves carry sympathetic fibers, except for the pelvic splanchnic nerves. The thoracic splanchnic nerves are recognised as medial branches from the lower seven thoracic sympathetic ganglia. They are pre-synaptic nerves of the sympathetic system, and include the GSN, the lesser splanchnic nerve, and the least splanchnic nerve. They pass through the diaphragm to send fibers to the celiac, aorticorenal, and superior mesenteric ganglia and plexuses. Further detail about the thoracic splanchnic nerves and the celiac ganglia are described in Loukas et al. (2010) Clinical Anatomy 23:512-22.

The GSN is derived from the fifth to ninth thoracic ganglia in humans, with the potential for contribution from the tenth thoracic ganglia. In most cases, the greater splanchnic nerve originates from four roots, before descending obliquely, giving off branches to the descending aorta and perforating the crus of the diaphragm. There are two GSNs in the human body and, while inhibition of either or both is possible according to the invention, the GSN of particular interest is the right GSN.

The GSN naturally carries sensory signals from various abdominal organs. By inhibiting neural activity in the GSN it is possible to achieve therapeutic effects, such as increasing glucose tolerance, thereby assisting in treating conditions associated with impaired glucose control.

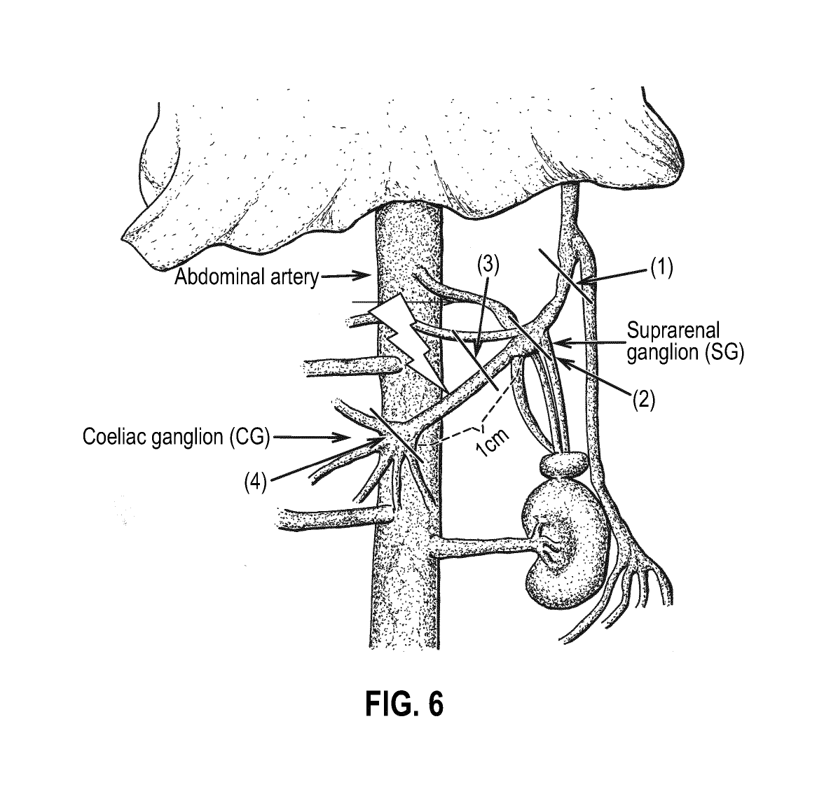

Although in principle the invention can inhibit activity at any point along the GSN, it is generally preferred to inhibit activity at or upstream of the celiac ganglion. In order to avoid unwanted physiological effects it is preferred to interrupt activity at or downstream of the suprarenal ganglion. Thus interruption between the suprarenal and celiac ganglia is preferred, and this region of the GSN is amenable to surgical intervention and electrode attachment (and, moreover, is more accessible than the carotid sinus nerve). Ideally, therefore, interruption of activity is localised to this region of the GSN. FIG. 6 illustrates positions of potential inhibition. Inhibition at (1) occurs after a branch in the GSN, but prior to the suprarenal ganglion, thus potentially affecting signalling to the vasculature which may not be desired. Inhibition at (2) is in the suprarenal ganglion, which is useful, but may affect signalling to the adrenal gland which, again, may not be desired, as it is preferred not to change catecholamine release. Position (4) applies inhibition to the celiac ganglion, which could inhibit other nerves that also contribute to the celiac ganglion and the celiac plexus. Thus inhibition between points (2) and (4) is preferred e.g. at (3), after the suprarenal ganglion but before the celiac ganglion. The lightning flash arrow in FIG. 6 shows where transections were made in the experiments described below, within a short stretch of the GSN which is about 1 cm long.

According to the invention, inhibition results in neural activity in at least part of the GSN being reduced compared to baseline neural activity in that part of the nerve. This reduction in activity can be across the whole nerve, in which case neural activity is reduced across the whole nerve. Thus inhibition may apply to both afferent and efferent fibers of the GSN, but in some embodiments inhibition may apply only to afferent fibers or only to efferent fibers. Results with resiniferatoxin suggest that inhibition of afferent GSN fibers is important for improving glucose tolerance, but the intraperitoneal data shown below suggest that efferent fibers (e.g. targeting the liver) may play an important role.

As used herein, “neural activity” of a nerve means the signalling activity of the nerve, for example the amplitude, frequency and/or pattern of action potentials in the nerve. The term “pattern”, as used herein in the context of action potentials in the nerve, is intended to include one or more of: local field potential(s), compound action potential(s), aggregate action potential(s), and also magnitudes, frequencies, areas under the curve and other patterns of action potentials in the nerve or sub-groups (e.g. fascicules) of neurons therein.

Modulation of neural activity, as used herein, is taken to mean that the signalling activity of the nerve is altered from the baseline neural activity—that is, the signalling activity of the nerve in the subject prior to any intervention. Modulation according to the present invention involves inhibition of GSN neural activity compared to baseline activity.

Inhibition may be partial inhibition. Partial inhibition may be such that the total signalling activity of the whole nerve is partially reduced, or that the total signalling activity of a subset of nerve fibres of the nerve is fully reduced (i.e. there is no neural activity in that subset of fibres of the nerve), or that the total signalling of a subset of nerve fibres of the nerve is partially reduced compared to baseline neural activity in that subset of fibres of the nerve. Inhibition of neural activity encompasses full inhibition of neural activity in the nerve—that is, embodiments where there is no neural activity in the whole nerve.

In some cases, the inhibition of neural activity may be a block of neural activity i.e. action potentials are blocked from travelling beyond the point of the block in at least a part of the GSN. A block on neural activity is thus understood to be blocking neural activity from continuing past the point of the block. That is, when the block is applied, action potentials may travel along the nerve or subset of nerve fibres to the point of the block, but not beyond the point of the block. Thus, the nerve at the point of block is modified in that the nerve membrane is reversibly depolarised or hyperpolarised by a signal (e.g. an electric field produced by an electrical signal), such that an action potential does not propagate through the modified nerve. Hence, the nerve at the point of the block is modified in that it has lost its capacity to propagate action potentials, whereas the portions of the nerve before and after the point of block have the capacity to propagate action potentials.

When an electrical signal is used with the invention, the block is based on the influence of electrical currents (e.g. charged particles, which may be one or more electrons in an electrode attached to the nerve, or one or more ions outside the nerve or within the nerve, for instance) on the distribution of ions across the nerve membrane.

At any point along the axon, a functioning nerve will have a distribution of potassium and sodium ions across the nerve membrane. The distribution at one point along the axon determines the electrical membrane potential of the axon at that point, which in turn influences the distribution of potassium and sodium ions at an adjacent point, which in turn determines the electrical membrane potential of the axon at that point, and so on. This is a nerve operating in is normal state, wherein action potentials propagate from point to adjacent point along the axon, and which can be observed using conventional experimentation. One way of characterizing a block of neural activity is a distribution of potassium and sodium ions at one or more points in the axon which is created not by virtue of the electrical membrane potential at adjacent a point or points of the nerve as a result of a propagating action potential, but by virtue of the application of a temporary external electrical field. The temporary external signal (e.g. the electrical field produced by the electrical signal) artificially modifies the distribution of potassium and sodium ions within a point in the nerve, causing depolarisation or hyperpolarisation of the nerve membrane that would not otherwise occur. The depolarisation or hyperpolarisation of the nerve membrane caused by the temporary external signal (e.g. the electrical field produced by the electrical signal) blocks the propagation of an action potential across that point, because the action potential is unable to influence the distribution of potassium and sodium ions, which is instead governed by the temporary external signal (e.g. the external electrical field produced by the electrical signal). This is a nerve operating in a disrupted state, which can be observed by a distribution of potassium and sodium ions at a point in the axon (the point which has been blocked) that has an electrical membrane potential that is not influenced or determined by the electrical membrane potential of an adjacent point.

In some embodiments the inhibition is a partial block; in other embodiments the inhibition is a full block. In a preferred embodiment, the inhibition is a partial or full block of neural activity in the GSN. Blocking may be a partial block, for example a reduction in neural activity of 5%, 10%, 15%, 20%, 25%, 30%, 35%, 40%, 45%, 40%, 50%, 60%, 70%, 80%, 90% or 95%, or blocking of neural activity in a subset of nerve fibres of the nerve. Alternatively, such blocking may be a full block—i.e. blocking of neural activity in the whole nerve.

Inhibition of neural activity may also be an alteration in the pattern of action potentials. It will be appreciated that the pattern of action potentials can be modulated without necessarily changing the overall frequency or amplitude. For example, Inhibition of the neural activity may be such that the pattern of action potentials is altered to more closely resemble a healthy state rather than a disease state.

Inhibition of neural activity may comprise altering the neural activity in various other ways, for example increasing or decreasing a particular part of the neural activity and/or stimulating new elements of activity, for example: in particular intervals of time, in particular frequency bands, according to particular patterns and so forth.

Inhibition of the neural activity may be temporary. As used herein, “temporary” is used interchangeably with “reversible”, each being taken to mean that the inhibition of neural activity is not permanent. That is, upon cessation of inhibition, neural activity in the nerve returns substantially towards baseline neural activity within 1-60 seconds, or within 1-60 minutes, or within 1-24 hours (e.g. within 1-12 hours, 1-6 hours, 1-4 hours, 1-2 hours), or within 1-7 days (e.g. 1-4 days, 1-2 days). In some instances of temporary inhibition, the neural activity returns substantially fully to baseline neural activity. That is, the neural activity following cessation of inhibition is substantially the same as the neural activity prior to inhibition (e.g. prior to a signal being applied).

In other embodiments, inhibition of the neural activity may be substantially persistent. As used herein, “persistent” is taken to mean that the inhibited neural activity has a prolonged effect. That is, upon cessation of inhibition, neural activity in the nerve remains substantially the same as when inhibition was occurring—i.e. the neural activity during and following inhibition is substantially the same.

Inhibition of the neural activity may be (at least partially) corrective. As used herein, “corrective” is taken to mean that the inhibited neural activity alters the neural activity towards the pattern of neural activity in a healthy individual. That is, upon cessation of inhibition, neural activity in the nerve more closely resembles (ideally, substantially fully resembles) the pattern of action potentials in the GSN observed in a healthy subject than prior to inhibition. Such corrective inhibition can be any inhibition as defined herein. For example, inhibition may result in a block on neural activity, and upon cessation of inhibition the pattern of action potentials in the nerve resembles the pattern of action potentials observed in a healthy subject. By way of further example, inhibition may result in neural activity resembling the pattern of action potentials observed in a healthy subject and, upon cessation of inhibition, the pattern of action potentials in the nerve remains the pattern of action potentials observed in a healthy subject.

By way of further example, inhibition may result in modulation such that GSN neural activity resembles the pattern of action potentials observed in a healthy subject, and upon cessation of the signal, the pattern of action potentials in the nerve resembles the pattern of action potentials observed in a healthy subject. It is hypothesised that such a corrective effect is the result of a positive feedback loop—that is, the underlying disease state is treated as result of the claimed methods, and therefore the chemosensory signals along the GSN are not abnormal, and therefore the disease state is not perpetuated by the abnormal GSN neural activity.

### Applying Signals to the GSN

Various methods can be used to inhibit neural activity in the GSN (e.g. see Luan et al. 2014, Front. Neuroeng. 7:27. doi:10.3389/fneng.2014.00027). Although severance or transection of the GSN can be used, the permanence of this procedure means that it is not preferred. Similarly, known techniques of splanchnicectomy (see Loukas et al. 2010) are also not preferred. Rather than using such destructive techniques, it is preferred to apply a signal to the GSN which results in the transfer of energy in a suitable form to carry out the intended effect of the signal. That is, application of a signal to the GSN may equate to the transfer of energy to (or from) the GSN to achieve the intended effect. For example, the energy transferred may be electrical, mechanical (including acoustic, such as ultrasound), electromagnetic (e.g. optical), magnetic or thermal energy. Application of a signal as used herein does not include a pharmacological intervention.

Signals applied according to the invention are ideally non-destructive. As used herein, a “non-destructive signal” is a signal that, when applied, does not irreversibly damage the underlying neural signal conduction ability of the nerve. That is, application of a non-destructive signal maintains the ability of the GSN (or fibres thereof, or other nerve tissue to which the signal is applied) to conduct action potentials when application of the signal ceases, even if that conduction is in practice inhibited or blocked as a result of application of the non-destructive signal.

Inhibition of GSN activity can be achieved using electrical signals. These will generally be applied via one or more transducers which are placed in signalling contact with the GSN. The electrical signal may be, for example, a voltage or current. In certain such embodiments the signal applied comprises a direct current (DC), such as a charge balanced direct current, or an alternating current (AC) waveform, or both a DC and an AC waveform. Characteristics of inhibitory electrical waveforms for use with the invention are described in more detail below. As used herein, “charge-balanced” in relation to a DC current is taken to mean that the positive or negative charge introduced into any system (e.g. a nerve) as a result of a DC current being applied is balanced by the introduction of the opposite charge in order to achieve overall (net) neutrality. A combination of charge balanced DC and AC is particularly useful, with the DC being applied for a short initial period after which only AC is used (e.g. see Franke et al. 2014, J Neural Eng 11(5):056012).

In certain embodiments, the electrical signal has a frequency of 0.5 to 100 kHz, optionally 1 to 50 kHz, optionally 5 to 50 KHz. In certain embodiments the signal has a frequency of 25 to 55 kHz, optionally 30-50 kHz. In certain embodiments, the signal has a frequency of 5-10 KHz. In certain embodiments, the electrical signal has a frequency of greater than 1 kHz. In certain embodiments, the electrical signal has a frequency of greater than 20 kHz, optionally at least 25 kHz, optionally at least 30 kHz. In certain embodiments the signal has a frequency of 30 kHz, 40 kHz or 50 kHz.

Before becoming inhibitory, electrical signalling can be preceded by a short period in which the nerve is instead stimulated (an “onset response” or “onset effect”). Various ways of avoiding an onset response are available. In certain embodiments, an onset response as a result of the signal being applied can be avoided if the signal does not have a frequency of 20 kHz or lower, for example 1-20 kHz, or 1-10 kHz. Frequency- and amplitude-transitioned waveforms to mitigate onset responses in high-frequency nerve blocking are described by Gerges et al. 2010 (J. Neural Eng. 7:066003). Amplitude ramping can also be used, as discussed by Bhadra et al. 2009 (DOI: 10.1109/IEMBS.2009.5332735), or a combination of KHFAC with charge balanced direct current waveforms can be used (Franke et al. 2014, J Neural Eng 11(5):056012). A combination of KHFAC and infra-red laser light (‘ACIR’) has also been used to avoid onset responses (Lothet et al. 2014, Neurophotonics 1(1):011010).

In certain embodiments the DC waveform or AC waveform may be a square, sinusoidal, triangular or complex waveform. The DC waveform may alternatively be a constant amplitude waveform. In certain embodiments the electrical signal is an AC sinusoidal waveform.

It will be appreciated by the skilled person that the current amplitude of an applied electrical signal necessary to achieve the intended neuromodulation will depend upon the positioning of the electrode and the associated electrophysiological characteristics (e.g. impedance). It is within the ability of the skilled person to determine the appropriate current amplitude for achieving the intended neuromodulation in a given subject. For example, the skilled person is aware of methods suitable to monitor the neural activity profile induced by neuromodulation.

In certain embodiments, the electrical signal has a current of 0.1-10 mA, optionally 0.5-5 mA, optionally 1 mA-2 mA, optionally 1 mA or 2 mA.

In certain embodiments, the signal is an electrical signal comprising an AC sinusoidal waveform having a frequency of greater than 25 kHz, optionally 30-50 kHz. In certain such embodiments, the signal can be an electrical signal comprising an AC sinusoidal waveform having a frequency of greater than 25 kHz, optionally 30-50 kHz having a current of 1 mA or 2 mA.

Some electrical forms of neuromodulation may use direct current (DC), or alternating current (AC) waveforms applied to a nerve using one or more electrodes. A DC block may be accomplished by gradually ramping up the DC waveform amplitude (Bhadra & Kilgore, IEEE Transactions on Neural systems and rehabilitation engineering, 2004 12:313-324).

Some other AC techniques include HFAC or KHFAC (high-frequency or kilohertz frequency) to provide a reversible block (for example see Kilgore & Bhadra, 2004, Medical and Biological Engineering and Computing, May; 42(3):394-406. Nerve conduction block utilising high-frequency alternating current). In the work of Kilgore & Bhadra, a proposed waveform was sinusoidal or rectangular at 3-5 kHz, and typical signal amplitudes that produced block were 3-5 Volts or 0.5-2.0 milliAmperes peak-to-peak. Further details of charge-balanced KHFAC, which can be used with the invention, are discussed by Kilgore & Bhadra (2014) Neuromodulation 17:242-55. Advantageously, KHFAC is reversible.

HFAC may typically be applied at a frequency of between 1 and 50 kHz at a duty cycle of 100% (Bhadra et al., Journal of Computational Neuroscience, 2007, 22:313-326). Methods for selectively blocking activity of a nerve by application of a waveform having a frequency of 5-10 kHz are described in U.S. Pat. No. 7,389,145. Similarly, U.S. Pat. No. 8,731,676 describes a method of ameliorating sensory nerve pain by applying a 5-50 kHz frequency waveform to a nerve.

Some commercially available nerve blocking systems include the Maestro™ system available from Enteromedics Inc. of Minnesota, USA. Similar neuromodulation devices are more generally discussed in US2014/0214129 and elsewhere.

The signal may comprise a mechanical signal. In certain embodiments, the mechanical signal is a pressure signal. In certain such embodiments, the transducer causes a pressure of at least 250 mmHg to be applied to the nerve, thereby inhibiting neural activity. In certain alternative embodiments, the signal is an ultrasonic signal. In certain such embodiments, the ultrasonic signal has a frequency of 0.5-2.0 MHz, optionally 0.5-1.5 MHz, optionally 1.1 MHz. In certain embodiments, the ultrasonic signal has a density of 10-100 W/cm², for example 13.6 W/cm² or 93 W/cm².

Another mechanical form of neuromodulation uses ultrasound which may conveniently be implemented using external instead of implanted ultrasound transducers.

The signal may comprise an electromagnetic signal, such as an optical signal. Optical signals can conveniently be applied using a laser and/or a light emitting diode configured to apply the optical signal. In certain such embodiments, the optical signal (for example the laser signal) has an energy density from 500 mW/cm² to 900 W/cm². In certain alternative embodiments, the signal is a magnetic signal. In certain such embodiments, the magnetic signal is a biphasic signal with a frequency of 5-15 Hz, optionally 10 Hz. In certain such embodiments, the signal has a pulse duration of 1-1000 μs, for example 500 μs.

Optogenetics is a technique in which genetically-modified cells express photosensitive features, which can then be activated with light to modulate cell function. Many different optogenetic tools have been developed for inhibiting neural firing. A list of optogenetic tools to suppress neural activity has been compiled (Ritter LM et al., 2014 Epilepsia doi: 10.1111/epi.12804.). Acrylamine-azobenzene-quaternary ammonium (AAQ) is a photochromic ligand that blocks many types of K+ channels and in the cis configuration, the relief of K+ channel block inhibits firing (Nat Neurosci. 2013 July; 16(7):816-23. doi: 10.1038/nn.3424. Optogenetic pharmacology for control of native neuronal signalling proteins. Kramer RH et al, which is incorporated herein by reference). Thus light can be used with genetic modification of target cells to achieve inhibition of neural activity, particularly in pre-clinical settings.

The signal may use thermal energy, and the temperature of a nerve can be modified to inhibit propagation of neurosignalling. For example, Patberg et al. (Blocking of impulse conduction in peripheral nerves by local cooling as a routine in animal experimentation. Journal of Neuroscience Methods 1984; 10:267-75, which is incorporated herein by reference) discuss how cooling a nerve blocks signal conduction without an onset response, the block being both reversible and fast acting, with onsets of up to tens of seconds. Heating the nerve can also be used to block conduction, and is generally easier to implement in a small implantable or localised transducer or device, for example using infrared radiation from laser diode or a thermal heat source such as an electrically resistive element, which can be used to provide a fast, reversible, and spatially very localised heating effect (see for example Duke et al. J Neural Eng. 2012 June; 9(3):036003. Spatial and temporal variability in response to hybrid electro-optical stimulation, which is incorporated herein by reference). Either heating, or cooling, or both could be conveniently provided in vivo using a Peltier element (see below).

Where the signal applied to a nerve is a thermal signal, the signal can reduce the temperature of the nerve. In certain such embodiments the nerve is cooled to 14° C. or lower to partially inhibit neural activity, or to 6° C. or lower, for example 2° C., to fully inhibit neural activity. In such embodiments, it is preferably not to cause damage to the nerve. In certain alternative embodiments, the signal increases the temperature of the nerve. In certain embodiments, neural activity is inhibited by increasing the nerve's temperature by at least 5° C., for example by 5° C., 6° C., 7° C., 8° C., or more. In certain embodiments, signals can be used to heat and cool a nerve simultaneously at different locations on the nerve, or sequentially at the same or different location on the nerve.

Inhibition can be applied to the GSN intermittently or continuously. Intermittent inhibition involves applying the inhibition in an (on-off)ₙ pattern, where n>1. For instance, inhibition can be applied continuously for at least 5 days, optionally at least 7 days, before ceasing for a period (e.g. 1 day, 2 days, 3 days, 1 week, 2 weeks, 1 month), before being again applied continuously for at least 5 days, etc. Thus inhibition is applied for a first time period, then stopped for a second time period, then reapplied for a third time period, then stopped for a fourth time period, etc. In such an embodiment, the first, second, third and fourth periods run sequentially and consecutively. The duration of the first, second, third and fourth time periods is independently selected. That is, the duration of each time period may be the same or different to any of the other time periods. In certain such embodiments, the duration of each of the first, second, third and fourth time periods may be any time from 1 second (s) to 10 days (d), 2 s to 7 d, 3 s to 4 d, 5 s to 24 hours (24 h), 30 s to 12 h, 1 min to 12 h, 5 min to 8 h, 5 min to 6 h, 10 min to 6 h, 10 min to 4 h, 30 min to 4 h, 1 h to 4 h. In certain embodiments, the duration of each of the first, second, third and fourth time periods is 5 s, 10 s, 30 s, 60 s, 2 min, 5 min, 10 min, 20 min, 30 min, 40 min, 50 min, 60 min, 90 min, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 2 d, 3 d, 4 d, 5 d, 6 d, 7 d.

In certain embodiments, inhibition is applied for a specific amount of time per day. In certain such embodiments, the signal is applied for 10 min, 20 min, 30 min, 40 min, 50 min, 60 min, 90 min, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h per day. In certain such embodiments, inhibition is applied continuously for the specified amount of time. In certain alternative such embodiments, inhibition may be applied discontinuously across the day, provided the total time of application amounts to the specified time.

Continuous inhibition may continue indefinitely, e.g. permanently. Alternatively, the continuous application may be for a minimum period, for example the signal may be continuously applied for at least 5 days, or at least 7 days.

Where inhibition is controlled by a device/system of the invention, and where a signal is continuously applied to the GSN, although the signal might be a series of pulses, the gaps between those pulses do not mean the signal is not continuously applied.

In certain embodiments, inhibition is applied only when the subject is in a specific state e.g. only when the subject is awake, only when the subject is asleep, prior to and/or after the ingestion of food, prior to and/or after the subject undertakes exercise, etc.

These various embodiments for timing of inhibition can all be achieved using the controller in a device/system of the invention.

### Conditions Associated with Impaired Glucose Control

The invention is useful for treating subjects suffering from conditions associated with impaired glucose control. Conditions associated with impaired glucose control include those conditions thought to cause the impairment (for example insulin resistance, obesity, metabolic syndrome, Type 1 diabetes, Hepatitis C infection, acromegaly) and conditions resulting from the impairment (for example obesity, sleep apnoea syndrome, dyslipidaemia, hypertension, Type 2 diabetes). It will be appreciated that some conditions can be both a cause of and caused by impaired glucose control. Other conditions associated with impaired with glucose control would be appreciated by the skilled person. It will also be appreciated that these conditions may also be associated with insulin resistance.

The invention is of particular interest in relation to insulin resistance, prediabetes, and type 2 diabetes.

As used herein, “impaired glucose control” is taken to mean an inability to maintain blood glucose levels at a normal level (i.e. within normal limits for a healthy individual). As will be appreciated by the skilled person, this will vary based on the type of subject and can be determined by a number of methods well known in the art, for example a glucose tolerance test (GTT). For example, in humans undergoing an oral glucose tolerance test, a glucose level at 2 hours of less than or equal to 7.8 mmol/L is considered normal. A glucose level at 2 hours of more than 7.8 mmol/L is indicative of impaired glucose control.

As used herein, “insulin resistance” has its normal meaning in the art i.e. in subject or patient exhibiting insulin resistance, the physiological response to insulin in the subject or patient is refractory, such that a higher level of insulin is required in order to control blood glucose levels, compared to the insulin level required in a healthy individual. Insulin sensitivity is used herein as the reciprocal to insulin resistance—that is, an increase in insulin sensitivity equates to a decrease in insulin resistance, and vice versa. Insulin resistance may be determined using any method known in the art, for example a GTT, a hyperinsulinaemic clamp or an insulin suppression test.

Treatment of the condition can be assessed in various ways, but typically involves an improvement in one or more detected physiological parameters. As used herein, an “improvement in a measurable physiological parameter” is taken to mean that, for any given physiological parameter, an improvement is a change in the value of that parameter in the subject towards the normal value or normal range for that value—i.e. towards the expected value in a healthy individual. For an example, in a subject having a condition associated with impaired glucose control (e.g. insulin resistance) an improvement in a measurable parameter may (depending on which abnormal values a subject is exhibiting) be one or more of: an increase in insulin sensitivity, a decrease in insulin resistance, a decrease in (fasting) plasma glucose concentration, a reduction in total fat mass, a reduction in visceral fat mass, a reduction in subcutaneous fat mass, a reduction in body mass index, aa reduction in obesity, a reduction in sympathetic tone, blood pressure, a reduction in plasma and/or tissue catecholamines, reduction in urinary metanephrines, and a reduction in glycated haemoglobin (HbA1c), and/or a reduction in circulating triglycerides. The invention might not lead to a change in all of these parameters.

In such embodiments, sympathetic tone is understood to be the neural activity in sympathetic nerves and/or associated sympathetic neurotransmitter measured in systemic or local tissue compartments in the sympathetic nervous system. Suitable methods for determining the value for any given parameter will be appreciated by the skilled person. By way of example, an increase in heart rate and/or blood pressure for a period at least 24 hrs is typically indicative of an increased sympathetic tone, as is aberrant heart rate variability, cardiac or renal norepinephrine spillover, skin or muscle microneurography and plasma/urine norepinephrine By way of further example, insulin sensitivity can be measured by the HOMA index or by a hyperinsulinemic clamp. By way of further example, total fat mass may be determined by bioimpedence. By way of further example, visceral fat can be indirectly determined by measuring abdominal perimeter. Further suitable methods for determining the value for any given parameter would be appreciated by the skilled person.

In certain embodiments of the invention, treatment of the condition is indicated by an improvement in the profile of neural activity in the GSN. That is, treatment of the condition is indicated by the neural activity in the GSN approaching the neural activity in a healthy individual.

Ideally, a subject displays an improvement in glucose tolerance as assessed by oral glucose tolerance test. Methods of the invention may be used to treat insulin resistance and T2D. The invention may also be used to treat metabolic syndrome.

As used herein, a physiological parameter is not affected by inhibition of GSN neural activity if the parameter does not change (in response to GSN activity inhibition) from the average value of that parameter exhibited by the subject or subject when no intervention has been performed i.e. it does not depart from the baseline value for that parameter.

The skilled person will appreciate that the baseline for any neural activity or physiological parameter in an individual need not be a fixed or specific value, but rather can fluctuate within a normal range or may be an average value with associated error and confidence intervals. Suitable methods for determining baseline values are well known to the skilled person.

As used herein, a measurable physiological parameter is detected in a subject when the value for that parameter exhibited by the subject at the time of detection is determined. A detector is any element able to make such a determination.

Thus, in certain embodiments, the invention further comprises a step of detecting one or more physiological parameters of the subject, wherein the signal is applied only when the detected physiological parameter meets or exceeds a predefined threshold value. In such embodiments wherein more than one physiological parameter is detected, the signal may be applied when any one of the detected parameters meets or exceeds its threshold value, alternatively only when all of the detected parameters meet or exceed their threshold values. In certain embodiments wherein the signal is applied by a neuromodulation device/system, the device/system further comprises at least one detector configured to detect the one or more physiological parameters.

In certain embodiments of the method, the one or more detected physiological parameters are one or more of the group consisting of: sympathetic tone, blood pressure, plasma insulin concentration, insulin sensitivity, plasma glucose concentration, glucose tolerance, total fat mass, visceral fat mass, plasma catecholamines (i.e. one or more of epinephrine, norepinephrine, metanephrine, normetanephrine and dopamine) content, tissue catecholamines content urinary metanephrines content, plasma HbA1c content and a reduction in circulating triglyceride concentration.

By way of example, a typical HbA1c content in a healthy human subject would be between 20-42 mmol/mol (4-6% of total Hb). An HbA1c content exceeding 42 mmol/mol may be indicative of a diabetic state.

In certain embodiments, the detected physiological parameter is an action potential or pattern of action potentials in a nerve of the subject, wherein the action potential or pattern of action potentials is associated with the condition associated with an impaired response to glucose that is to be treated. In certain such embodiments, the nerve is a sympathetic nerve.

A “predefined threshold value” for a physiological parameter is the minimum (or maximum) value for that parameter that must be exhibited by a subject or subject before the specified intervention is applied. For any given parameter, the threshold value may be defined as a value indicative of a pathological state or a disease state (e.g. sympathetic tone (neural, hemodynamic (e.g. heart rate, blood pressure, heart rate variability) or circulating plasma/urine biomarkers) greater than a threshold sympathetic tone, or greater than a sympathetic tone in a healthy individual, blood insulin levels greater than healthy levels, GSN signalling exhibiting a certain activity level or pattern). Alternatively, the threshold value may be defined as a value indicative of a physiological state of the subject (that the subject is, for example, asleep, post-prandial, or exercising). Appropriate values for any given parameter would be simply determined by the skilled person (for example, with reference to medical standards of practice).

Such a threshold value for a given physiological parameter is exceeded if the value exhibited by the subject is beyond the threshold value—that is, the exhibited value is a greater departure from the normal or healthy value for that parameter than the predefined threshold value.

In certain embodiments of the method, the method does not affect the cardiopulmonary regulation function of the GSN. In particular embodiments, the method does not affect one or more physiological parameters in the subject selected from the group consisting of: pO₂, pCO₂, blood pressure, oxygen demand and cardio-respiratory responses to exercise and altitude. Suitable methods for determining the value for any given parameter would be appreciated by the skilled person.

A subject of the invention may, in addition to having an implant, receive medicine for their condition. For instance, a subject having an implant according to the invention may receive a diabetes medicine (which will usually continue medication which was occurring before receiving the implant). Such medicines include, but are not limited to: metformin; sulfonylureas, such as glyburide, glipizide, or glimepiride; meglitinides, such as repaglinide or nateglinide; thiazolidinediones, such as rosiglitazone or pioglitazone; DPP-4 inhibitors, such as sitagliptin, vildagliptin, saxagliptin or linagliptin; GLP-1 receptor agonists, such as exenatide or liraglutide; SGLT2 inhibitors, such as canagliflozin or dapagliflozin. Thus the invention provides the use of these medicines in combination with a device/system of the invention.

### Devices and System for Implementing the Invention

The invention can be implemented using a device or system which can inhibit neural activity within the GSN. Such a device/system can comprise (i) one or more transducers configured to apply a signal to the GSN and (ii) a controller coupled to the one or more transducers, the controller controlling the signal to be applied by the one or more transducers, such that the signal can be applied to inhibit neural activity of the GSN to produce the desired physiological response in the subject

The various components are preferably part of a single physical device. As an alternative, however, the invention may use a system in which the components are physically separate, and communicate wirelessly. Thus, for instance, the transducer and the controller can be part of a unitary device, or together may form a system (and, in both cases, further components may also be present to form a larger device or system e.g. a power source, a sensor, etc.).

Devices/systems of the invention are configured to modulate neural activity of the GSN. Neuromodulation devices/systems as described herein can be comprised of one or more parts. The neuromodulation devices/systems comprise at least one transducer capable of effectively applying a signal to a nerve. In those embodiments in which the neuromodulation device/system is at least partially implanted in the subject, the elements of the device/system that are to be implanted in the subject are constructed such that they are suitable for such implantation. Such suitable constructions would be well known to the skilled person.

Various exemplary fully implantable neuromodulation devices are currently available, such as the vagus nerve stimulator of SetPoint Medical, in clinical development for the treatment of rheumatoid arthritis (Arthritis & Rheumatism, Volume 64, No. 10 (Supplement), page S195 (Abstract No. 451), October 2012. “Pilot Study of Stimulation of the Cholinergic Anti-Inflammatory Pathway with an Implantable Vagus Nerve Stimulation Device in Patients with Rheumatoid Arthritis”, Frieda A. Koopman et al), and the INTERSTIM™ device (Medtronic, Inc.), a fully implantable device utilised for sacral nerve modulation in the treatment of overactive bladder.

Suitable neuromodulation devices/systems can be fabricated with characteristics as described herein, for example for implantation within the nerve (e.g. intrafascicularly), for partially or wholly surrounding the nerve (e.g. a cuff interface with the nerve).

As used herein, “implanted” is taken to mean positioned within the subject's body. Partial implantation means that only part of the device/system is implanted—i.e. only part of the device/system is positioned within the subject's body (typically in proximity to the GSN), with other elements of the device/system external to the subject's body. For example, the transducer and controller of the device/system may be wholly implanted within the subject, and an input element may be external to the subject's body. Wholly implanted means that the entire of the device/system is positioned within the subject's body e.g. fully beneath the subject's skin. Parts of the device/system, for example the transducer and the controller, may be suitable to be wholly implanted in the subject such that the signal can be applied to the GSN, and other parts of the device/system may be external to the body, for example an input element or remote charging element. In certain embodiments, the device/system is suitable to be wholly implanted in the subject.

In those embodiments in which the device/system has at least two transducers, the signal which each of the transducers is configured to apply can be independently selected from an electrical signal, an optical signal, an ultrasonic signal, and a thermal signal. That is, each transducer may be configured to apply a different signal. Alternatively, in certain embodiments each transducer is configured to apply the same signal.

In certain embodiments, each of the one or more transducers may be comprised of one or more electrodes, one or more photon sources, one or more ultrasound transducers, one more sources of heat, or one or more other types of transducer arranged to put the signal into effect. Characteristics of the signals to be applied to a nerve to inhibit its activity are discussed above, and a device/system of the invention will be implemented accordingly.

In embodiments where electrical signals are applied to a nerve, the transducer(s) in a device/system will include electrode(s). Such electrodes may be bipolar or tripolar electrodes. An electrode may be a cuff electrode or a wire electrode.

In certain such embodiments, all the transducers are electrodes configured to apply an electrical signal, optionally the same electrical signal.

In embodiments in which the applied signal is a thermal signal, at least one of the one or more transducers is a transducer configured to apply a thermal signal. In certain such embodiments, all the transducers are configured to apply a thermal signal, optionally the same thermal signal. In these embodiments, one or more transducers may comprise a Peltier element configured to apply a thermal signal. Optionally all of the one or more transducers can comprise a Peltier element. In certain embodiments, one or more transducer can comprise a laser diode configured to apply a thermal signal, optionally all of the one or more transducers comprise a laser diode configured to apply a thermal signal. In certain embodiments, one or more transducer can comprise an electrically resistive element configured to apply a thermal signal.

In certain embodiments, one or more of the one or more transducers comprise a Peltier element configured to apply a thermal signal, optionally all of the one or more transducers comprise a Peltier element. In certain embodiments, one or more of the one or more transducers comprise a laser diode configured to apply a thermal signal, optionally all of the one or more transducers comprise a laser diode configured to apply a thermal signal. In certain embodiments, one or more of the one or more transducers comprise an electrically resistive element configured to apply a thermal signal, optionally all of the one or more transducers comprise an electrically resistive element configured to apply a thermal signal.

In certain embodiments, the device/system further comprises one or more of a power supply element, for example a battery, and/or one or more communication elements. The device/system may be powered by inductive powering or a rechargeable power source.

In certain embodiments, a device/system of the invention further comprises means to detect one or more physiological parameters in the subject. Such a means may be one or more detectors configured to detect the one or more physiological parameters. That is, in such embodiments each detector may detect more than one physiological parameter, for example all the detected physiological parameters. Alternatively, in such embodiments each detector is configured to detect a separate parameter of the one or more physiological parameters detected.

In such certain embodiments, the controller is coupled to the means to detect one or more physiological parameters, and causes the transducer or transducers to apply the signal when the physiological parameter is detected to be meeting or exceeding a predefined threshold value.

In certain embodiments, the one or more detected physiological parameters comprise one or more of the group consisting of: sympathetic tone, blood pressure, plasma insulin concentration, plasma glucose concentration, plasma catecholamine concentration (i.e. one or more of epinephrine, norepinephrine, metanephrine, normetanephrine and dopamine) concentration, tissue catecholamine concentration, plasma HbA1c concentration or plasma triglyceride concentration.

In certain embodiments, the one or more detected physiological parameters comprise an action potential or pattern of action potentials in a nerve of the subject, wherein the action potential or pattern of action potentials is associated with the condition associated with an impaired response to glucose that is to be treated.

It will be appreciated that any two or more of the indicated physiological parameters may be detected in parallel or consecutively. For example, in certain embodiments, the controller is coupled to a detector or detectors configured to detect the pattern of action potentials in the GSN at the same time as glucose tolerance in the subject.

In some embodiments, the device/system further comprises an input means. This permits the status of the subject (e.g. whether the subject is awake, asleep, pre- or post-eating, or pre- or post-taking exercise) to be input into the device/system by the subject or by a physician. In alternative embodiments, the device/system further comprises a detector configured to detect the status of the subject. In all of these embodiments, the device/system may be programmed to apply its signal to the GSN only when the subject is in a particular state (e.g. only when awake).

A device/system of the invention is preferably made from, or coated with, a biostable and biocompatible material. This means that the device/system is both protected from damage due to exposure to the body's tissues and also minimises the risk that the device/system elicits an unfavourable reaction by the host (which could ultimately lead to rejection). The material used to make or coat the device/system should ideally resist the formation of biofilms. Suitable materials include, but are not limited to, poly(p-xylylene) polymers (known as Parylenes) and polytetrafluoroethylene.

A device/system of the invention will generally weigh less than 50 g.

### Implanting a Device/System of the Invention

The invention provides a method of implanting a device/system of the invention in a subject, comprising a step of: positioning at least one transducer of the device/system in signalling contact with the subject's GSN. In some embodiments the method includes a step of activating the device/system; in other methods, this activation step does not occur.

The term “signalling contact” means that a transducer's signal (whether electrical, thermal, etc.) is sufficiently near the GSN that it can cause the desired change in the GSN's function. In some embodiments the transducer may be attached directly to the GSN, but in other embodiments it may be near to or surrounding the GSN.

Activation of the device/system means that it is placed into a state where it can cause the desired change in the GSN's function. In some embodiments the device/system is positioned such that it is in signalling contact with the GSN, but such signalling cannot occur because the device/system is not activated. In such embodiments the subject obtains no therapeutic benefit from the device/system's presence. Only after activation is such benefit achieved. The device/system is activated when the device/system is in an operating state such that the signal will be applied to the GSN e.g. as determined by the device/system's controller.

A device/system of the invention can be implanted into a subject either partially or fully. Full implantation is preferred, as discussed above.

### Implementation of the Invention

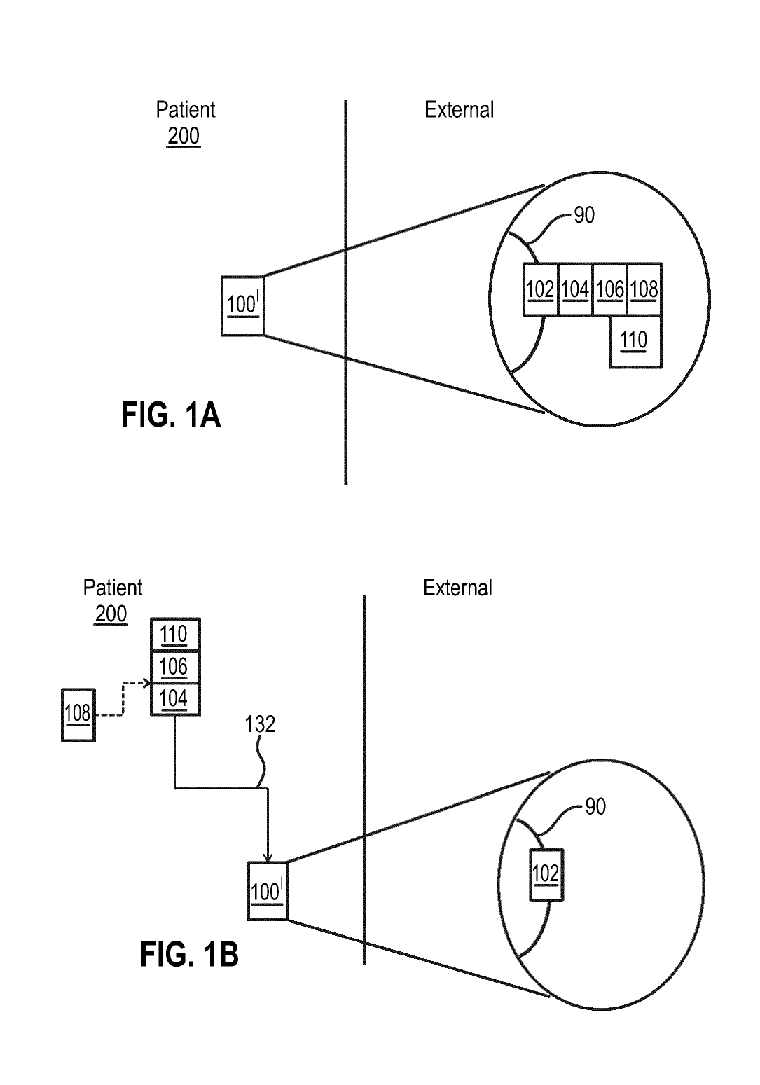

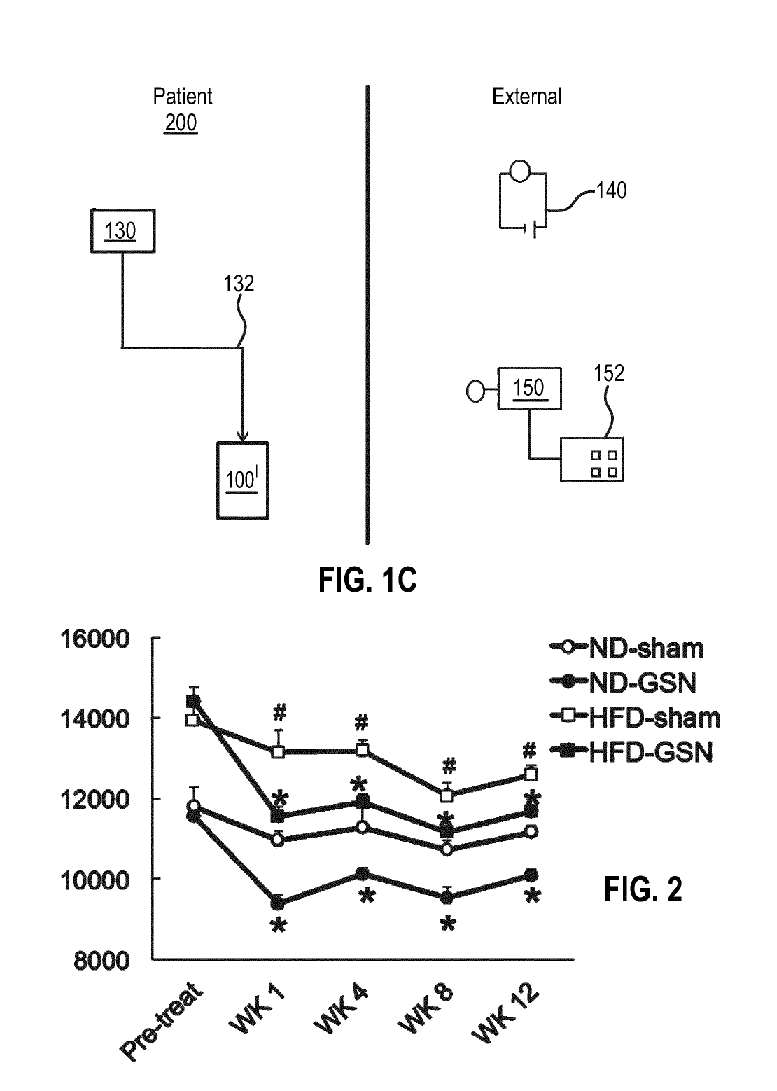

Implementation of all aspects of the invention (as discussed both above and below) will be further appreciated by reference to FIGS. 1A-1C.

FIGS. 1A-1C show how the invention may be put into effect using one or more neuromodulation devices which are implanted in, located on, or otherwise disposed with respect to a subject 200 in order to carry out any of the various methods described herein. In this way, one or more neuromodulation devices can be used to treat a condition associated with impaired glucose control in a subject, by modulating GSN neural activity. The components are shown as being connected within a single device, but they could be separate to form a system which performs the same function, with the components communicating wirelessly.

In each of the FIGS. 1A-1C a neuromodulation device 100 is fully or partially implanted in the subject, or otherwise located, so as to provide neuromodulation of the GSN. FIG. 1A also shows schematically components of one of the neuromodulation devices 100, in which the device comprises several elements, components or functions grouped together in a single unit and implanted in the subject 200. A first such element is a transducer 102 which is shown in proximity to a carotid sinus nerve 90 of the subject. The transducer 102 may be operated by a controller element 104. The device may comprise one or more further elements such as a communication element 106, a detector element 108, a power supply element 110 and so forth.

The neuromodulation device may carry out the required neuromodulation independently, or in response to one or more control signals. Such a control signal may be provided by the controller 104 according to an algorithm, in response to output of one or more detector elements 108, and/or in response to communications from one or more external sources received using the communications element. As discussed herein, the detector element(s) could be responsive to a variety of different physiological parameters.

FIG. 1B illustrates some ways in which the device of FIG. 1A may be differently distributed. For example, in FIG. 1B the neuromodulation device 100 comprises transducers 102 implanted proximally to the GSN 90, but other elements such as a controller 104, a communication element 106 and a power supply 110 are implemented in a separate control unit 130 which may also be implanted in, or carried by the subject. The control unit 130 then controls the transducers in the neuromodulation device via connections 132 which may for example comprise electrical wires and/or optical fibres for delivering signals and/or power to the transducers.

In the arrangement of FIG. 1B one or more detectors 108 are located separately from the control unit, although one or more such detectors could also or instead be located within the control unit 130 and/or in the neuromodulation device 100. The detectors may be used to detect one or more physiological parameters of the subject, and the controller element or control unit then causes the transducers to apply the signal in response to the detected parameter(s), for example only when a detected physiological parameter meets or exceeds a predefined threshold value. Physiological parameters which could be detected for such purposes include sympathetic tone, blood pressure, plasma insulin concentration, insulin sensitivity, plasma glucose concentration, glucose tolerance, plasma catecholamine concentration, tissue catecholamine concentration, plasma HbA1c concentration and plasma triglyceride concentration. Similarly, a detected physiological parameter could be an action potential or pattern of action potentials in a nerve of the subject, for example an efferent or more particularly a sympathetic nerve, wherein the action potential or pattern of action potentials is associated with the condition to be treated. The or each detector 108 may be located on or proximal to the GSN, such as to detect the action potential or pattern of action potentials in the GSN, as indicative of a disease state.

A variety of other ways in which the various functional elements could be located and grouped into the neuromodulation devices, a control unit 130 and elsewhere are of course possible. For example, one or more sensors of FIG. 1B could be used in the arrangement of FIG. 1A or 1C or other arrangements.

FIG. 1C illustrates some ways in which some functionality of the device of FIG. 1A or 1B is provided not implanted in the subject. For example, in FIG. 1C an external power supply 140 is provided which can provide power to implanted elements of the device in ways familiar to the skilled person, and an external controller 150 provides part or all of the functionality of the controller 104, and/or provides other aspects of control of the device, and/or provides data readout from the device, and/or provides a data input facility 152. The data input facility could be used by a subject or other operator in various ways, for example to input data relating to the subject's current or expected activities such as sleep, eating, or physical exertion.

Each neuromodulation device may be adapted to carry out the neuromodulation required using one or more physical modes of operation which typically involve applying a signal to the GSN, such a signal typically involving a transfer of energy to (or from) the body or nerve. As already discussed, such modes may comprise modulating the GSN using an electrical signal, an optical signal, an ultrasound or other mechanical signal, a thermal signal, a magnetic or electromagnetic signal, or some other use of energy to carry out the required modulation. Such signals may be non-destructive signals. Such modulation may comprise increasing, inhibiting, or otherwise changing the pattern of neural activity in the GSN. To this end, the transducer 90 illustrated in FIG. 1A could be comprised of one or more electrodes, one or more photon sources, one or more ultrasound transducers, one more sources of heat, or one or more other types of transducer arranged to put the required neuromodulation into effect.

The neural modulation device(s) may be arranged to inhibit neural activity of the GSN by using the transducer(s) to apply an electrical signal, for example a voltage or current, for example a direct current (DC) such as a charge balanced direct current, or an AC waveform, or both. In such embodiments, the transducers configured to apply the electrical signal are electrodes.

Although the invention is generally applicable, as explained above, for treating various disorders in various ways, there are some clear preferences. In particular: the subject is ideally a human being; the subject should be suffering from insulin resistance, prediabetes or T2D; inhibition of GSN signalling is achieved using a device of the invention which is partially or fully implanted in the subject; and/or inhibitory signals are applied to the GSN between the suprarenal and celiac ganglia.

### General

The terms as used herein (both above and below) are given their conventional meaning in the art as understood by the skilled person, unless otherwise defined above. In the case of any inconsistency or doubt, a definition provided herein should take precedence.

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

Unless otherwise indicated each embodiment as described herein may be combined with another embodiment as described herein.

### Modes for Carrying Out the Invention

### Effect of GSN Transection on Oral Glucose Tolerance

Male Sprague-Dawley rats were fed a high fat diet (HFD; 60% calories by fat) or a normal diet (ND). After 7 weeks, under anesthesia with 2% isoflurane, the left or right splanchnic nerve were exposed via a dorsolateral incision and left or right (respectively) subcostal muscle penetration. The greater splanchnic nerve (GSN) branch, just below the suprarenal ganglion, was then transected with microscissors. Sham operation was performed by exposing and visualizing the splanchnic nerve, without transection. Finally, dorsolateral incision was closed.

Various physiological parameters were examined. In particular, an oral glucose tolerance test (OGTT) was conducted before (pre) and 1, 4, 8 and 12 weeks after GSN-ectomy. Rats were fasted overnight prior to blood glucose test with free access to water. On the testing day, a baseline blood glucose concentration (0 min time point) was measured from a tail snap using a glucose matter (Bayer HealthCare LLC). At same time, ˜50 μl of blood was been collected and kept in ice for insulin test later. 10% glucose solution was then be orally administrated (1 g/kg, 10 ml/kg). The blood glucose levels were measured and blood was collected at 30, 60, 90 and 120 min after the glucose administration. All of the tests were been performed between 10 am & 2 pm. The results in FIG. 2 show that, in both the ND and HFD groups, rats with the GSN-ectomy showed lower OGTT scores than the sham-treated rats, with statistical significance.

Other physiological measurements and biomarker measurements did not show any significant differences between the groups. For instance, GSN-ectomy failed to show any significant effect on systolic and diastolic blood pressure over the 12 week monitoring period (except for diastolic pressure at week 8).

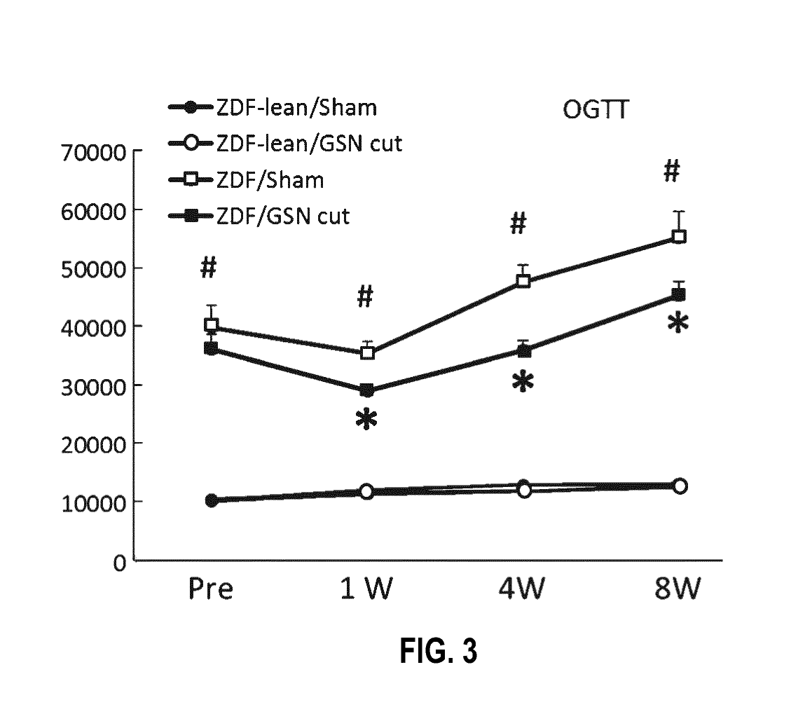

Similar experiments were performed in Zucker Diabetic Fatty (ZDF) or Zucker Lean (ZL) rats fed on a normal diet. FIG. 3 shows no difference in OGTT scores between the GSN-ectomy and sham-treated ZL rats, but in ZDF rats the GSN-ectomy led to a statistically significant reduction in OGTT score at all time points after the operation, with a reduction of ˜20-25% after 12 weeks.

### Effect of GSN Transection on Intraperitoneal Glucose Tolerance

The Zucker rats were also tested for glucose tolerance in response to intraperitoneal glucose. Rats were fasted overnight prior to a blood glucose test with free access to water. On the testing day, a baseline blood glucose concentration (0 min time point) was measured using a glucose meter, and rats were then injected with 50% glucose (1 g/kg body weight, 2 ml/kg). Blood glucose levels were measured 10, 30, 60, 90 and 120 min after the glucose injection.

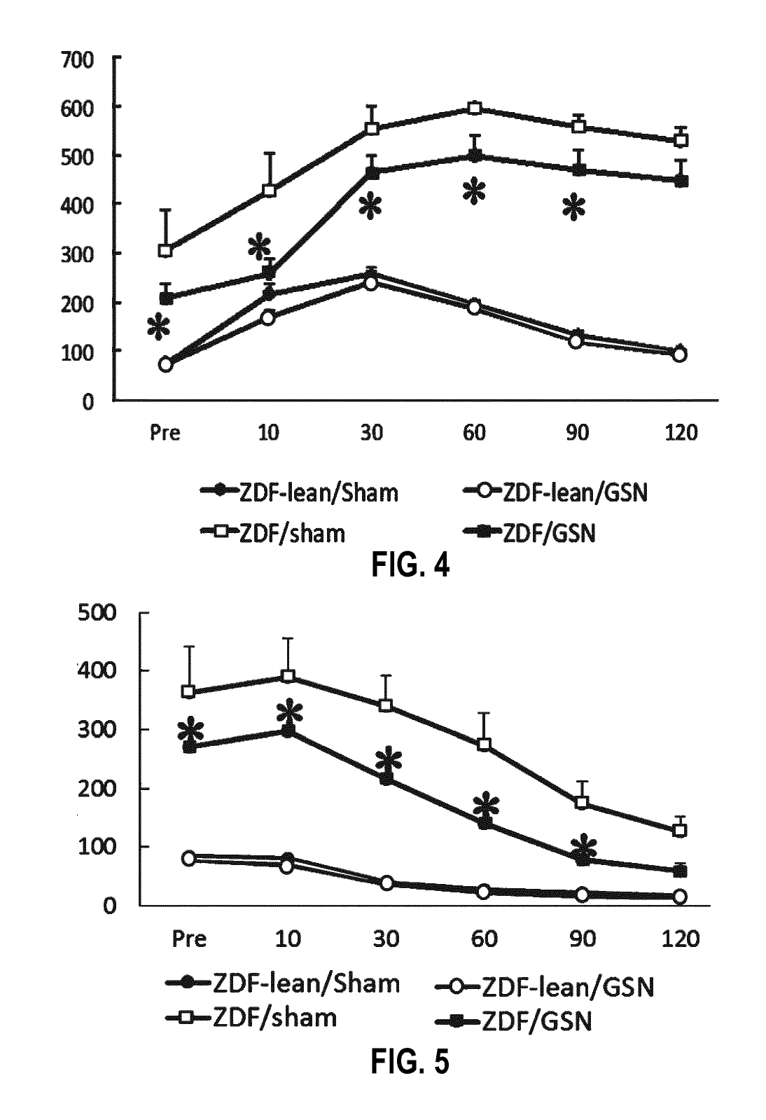

FIG. 4 shows that the ZL rats responded in the same way regardless of GSN-ectomy. In contrast, blood glucose levels in ZDF rats were significantly lower in the GSN-ectomy group for up to 90 minutes. Because the i.p. route avoids the stomach and duodenum, these results show that the OGTT results were not dependent on an incretin-like effect, and suggest that the effect on glucose metabolism is mediated by the liver rather than the gut.

Insulin tolerance testing was also performed. Rats were fasted overnight with free access to water prior to blood glucose testing. On the testing day, after a baseline glucose measurement, rats were injected i.p. with a standard dose of insulin (0.75 U/kg body weight). Blood glucose levels were measured 10, 30, 60, 90 and 120 min after the injection of insulin.

FIG. 5 shows that significantly lower glucose levels were achieved ZDF/GSN-ectomy rats when compared to the sham-treated ZDF rats, whereas there was no effect of GSN-ectomy in the ZL rats.

### Effects on Blood Pressure

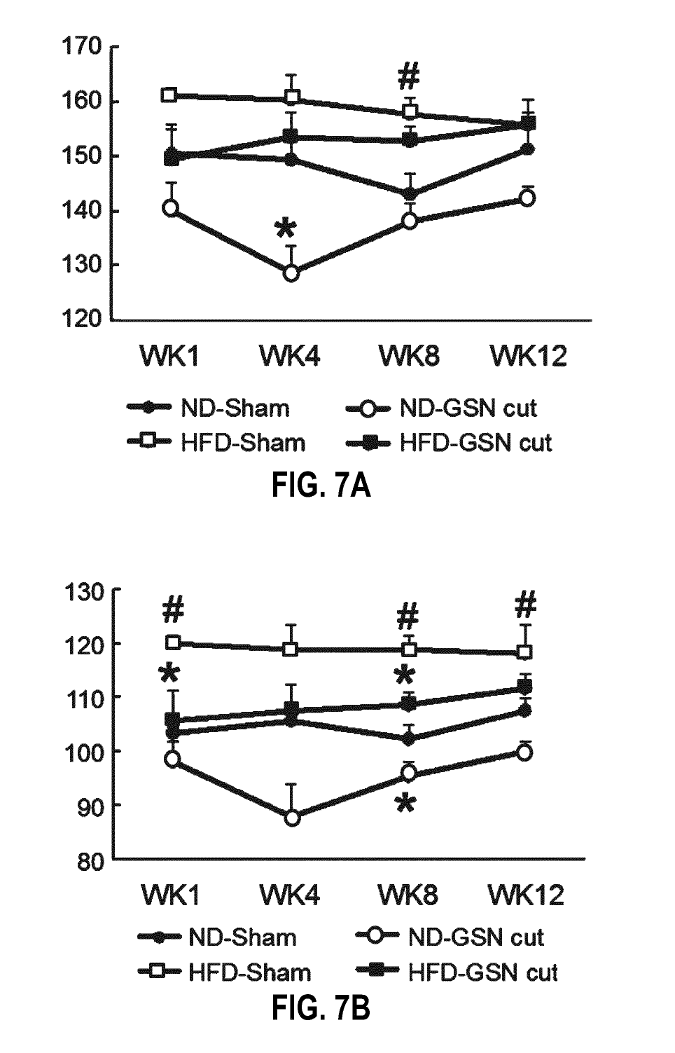

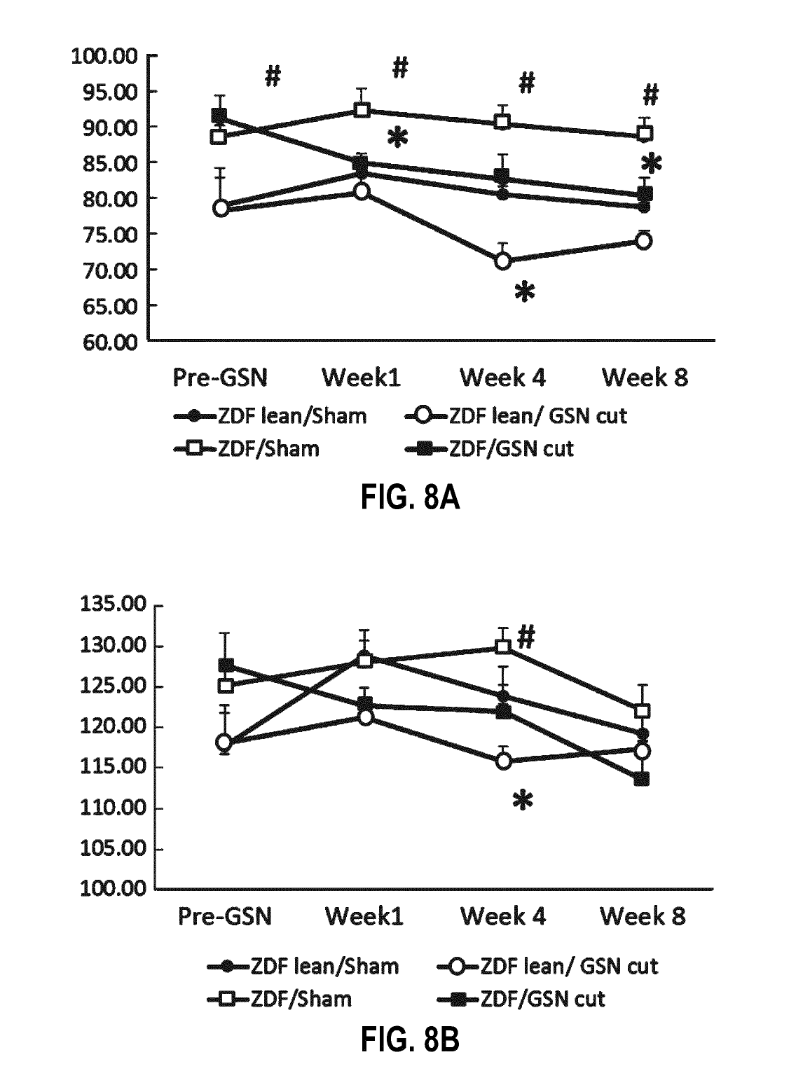

GSN interruption also caused a significant change in mean blood pressure in HFD rats (but not in ND rats) at 1 week, an effect that was sustained up to 8 weeks (FIG. 7). In ZDF rats, both diastolic and mean blood pressure were significantly lower at 8 weeks in the GSN-ectomy rats, but no difference was seen in the two lean groups (FIG. 8). This effect on blood pressure can be a further advantage in the context of metabolic syndrome.

### Conclusions

Duodenal innervation plays a role in the pathogenesis of insulin resistance and obesity-induced T2D, thus providing a rationale for using bioelectronics (or other neuromodulatory approaches) to inhibit GSN activity and thereby assist in diabetes therapy.

The foregoing detailed description has been provided by way of explanation and illustration, and is not intended to limit the scope of the appended claims. Many variations in the presently preferred embodiments illustrated herein will be apparent to one of ordinary skill in the art, and remain within the scope of the appended claims and their equivalents. Thus it will be understood that the invention is described above by way of example only and modifications may be made whilst remaining within the scope and spirit of the invention.

1.-3. (canceled)

4. A device or system for inhibiting the neural activity of a subject's greater splanchnic nerve (GSN), the device or system comprising:
(i) one or more transducers configured to apply a signal to the GSN of the subject, optionally at least two such transducers; and
(ii) a controller coupled to the one or more transducers, the controller controlling the signal to be applied by the one or more transducers, such that the signal inhibits the neural activity of the GSN to produce a physiological response in the subject, wherein the physiological response is one or more of the group consisting of: an increase in insulin sensitivity in the subject, an improvement in glucose tolerance in the subject, a decrease in fasting plasma glucose concentration in the subject, a reduction in subcutaneous fat content in the subject, and/or a reduction in obesity in the subject.

5. (canceled)

6. A device or system according claim 4, wherein the signal is an electrical signal, an optical signal, an ultrasonic signal, or a thermal signal.

7. A device or system according to claim 4, wherein the signal is an electrical signal, and each transducer configured to apply the signal is an electrode.

8. (canceled)

9. A device or system according to claim 7, wherein the signal comprises an alternating current (AC) waveform of greater than 1 kHz frequency.

10. A device or system according to claim 9, wherein the AC waveform has a frequency of greater than 20 kHz.

11. (canceled)

12. A device or system according to claim 7, wherein the electrical signal has a current of 0.5-5 mA.

13.-17. (canceled)

18. A device or system according to claim 4 wherein the inhibition in neural activity as a result of applying the signal is a partial block or a full block of neural activity in the GSN.

19.-24. (canceled)

25. A method of inhibiting neural signalling in the GSN of a subject comprising: (i) implanting in the subject a device/system according to claim 4; (ii) positioning at least one transducer of the device/system in signalling contact with the subject's GSN; and (iii) activating the device/system.

26.-29. (canceled)

30. A method of treating at least one condition associated with impaired glucose control in a subject, wherein the condition is at least one of the group consisting of insulin resistance, prediabetes, type 2 diabetes, and metabolic syndrome, the method comprising applying a signal to a part or all of the GSN of said subject to inhibit the neural activity of said GSN.

31. A method according claim 30, wherein treatment of the condition is indicated by an improvement in a measurable physiological parameter, wherein said measurable physiological parameter is at least one of the group consisting of: sympathetic tone, insulin sensitivity, glucose sensitivity, (fasting) glucose concentration, total fat mass, visceral fat mass, subcutaneous fat mass, plasma catecholamines, urinary metanephrines, and glycated haemoglobin (HbA1c).

32. A method according to claim 30, wherein the inhibition in neural activity as a result of applying the signal is a partial block or a full block of neural activity in the GSN.

33.-39. (canceled)

40. A method according to claim 30, wherein the signal is an electrical current and, when the signal is applied by a neuromodulation device or system, each transducer configured to apply the signal is an electrode.

41. A method according to claim 40, wherein the signal comprises an alternating current (AC) waveform of greater than 1 kHz frequency.

42. A method according to claim 41, wherein the AC waveform has a frequency of greater than 20 kHz.

43.-44. (canceled)

45. A method according to claim 40, wherein the electrical signal has a current of 0.5-5 mA.

46-59. (canceled)
